(19) United States
(12) Patent Application Publication
(10) Pub. No.: US 2013/0065234 A1
(43) Pub. Date: Mar. 14, 2013

(54) METHOD FOR DIAGNOSING BONE AND JOINT DISEASE BASED ON SINGLE NUCLEOTIDE POLYMORPHISM IN CHROMOSOME 10Q24

(71) Applicant: Shiro Ikegawa, Yokohama-shi (JP)

(72) Inventor: Shiro Ikegawa, Yokohama-shi (JP)

(73) Assignee: RIKEN, Saitama (JP)

(21) Appl. No.: 13/589,420

(22) Filed: Aug. 20, 2012

(30) Foreign Application Priority Data
Aug. 22, 2011 (JP) 2011-180578

(51) Int. Cl. C12Q 1/68; C07H 21/04

(52) U.S. Cl. 435/6.11; 536/24.31; 536/24.33

(57) ABSTRACT

Provided is a method for diagnosing a bone and joint is disease such as scoliosis. A single nucleotide polymorphism present in region 24 on the long arm of chromosome 10 (region 10q24) is analyzed and the risk of onset of a bone and joint disease and/or the presence or absence of onset of the same are diagnosed on the basis of a result of the analysis.

### TECHNICAL FIELD

The present invention relates to a diagnosis method for determining the risk of onset of a bone and joint disease such as scoliosis and/or the presence or absence of onset of the same and to a reagent used for the diagnosis method.

### BACKGROUND ART

Scoliosis is a disease condition characterized by a lateral curvature of the spine. A type of scoliosis whose exact etiology is unknown is called idiopathic scoliosis, which accounts for 80 to 90% of scoliosis cases.

Scoliosis occurs often in school-age children, particularly in girls. Adolescent idiopathic scoliosis (AIS) is defined by a curvature with Cobb angle, an indicator of scoliosis, of at least 10° that occurs in children between age 10 to skeletal maturity and whose etiology is not clear. The frequency of onset of AIS in school-age children is 2% in Japan and 2 to 3% in the world. In Japan, about 10,000 people develop AIS every year.

Diagnosis of scoliosis is made mainly by X-ray examination. X-ray examination is, however, not useful for preclinical or early diagnosis of scoliosis. In addition, idiopathic scoliosis is treated only by symptomatic therapy. Since the etiology thereof is unclear, no causal treatment has been established. Therefore, to allow for the preclinical diagnosis (risk diagnosis) and early diagnosis of scoliosis and allow for the causal treatment thereof, identifications of genes and single nucleotide polymorphisms (SNPs) associated with scoliosis are desired.

Many genetic loci causing AIS have been discovered by genome-wide linkage analysis, and AIS has been thought to be due to complicated genetic predisposition (Wise C. A. et al., Curr. Genomics 9, 51-59 (2008); Raggio, C. L. et al., J. Orthop. Res. 27, 1366-1372 (2009)). Additionally, AIS-susceptibility genes have been reported by candidate gene analysis (Wu, J. et al. Spine 31, 1131-1136 (2006); Zhang, H. Q. et al. Spine 34, 760-764 (2009); Chen, Z. et al. Eur. J. Hum. Genet. 17, 525-532 (2009); Qiu, X. S. et al. Spine 32, 1748-1753 (2007); Wang, H. et al. Spine 33, 2199-2203 (2008); Inoue, M. et al. Stud. Health Technol. Inform. 91, 90-96 (2002); and Yeung, H. Y. et al. Stud. Health Technol. Inform. 123, 18-24 (2006)). However, none of the genes has shown replicated association with AIS in subjects of other races (Takahashi, Y. et al. J. Orthop. Res. 29, 834-837 (2011), Takahashi, Y. et al. J. Orthop. Res. (2011)).

Furthermore, recently, Candidate AIS-associated genes have been reported by a genome-wide association study (GWAS) based on the transmission disequilibrium test (TDT) (Sharma, S. et al. Hum Mol Genet. 20, 1456-1466 (2011)). However, the associations of the genes with AIS were not replicated in case-control association analysis after multiple testing corrections.

As described above, despite the presence of findings about scoliosis-susceptibility genes, there have been no reports of genes and single nucleotide polymorphisms (SNPs) whose associations with scoliosis were confirmed at genome-wide level.

LBX1 gene present in region 24 on the long arm of chromosome 10 (10q24) has been cloned as a homeobox gene and there have been reported the expressions of the gene in dorsal spinal cord, hindbrain, a part of cardiac neural crest, muscle precursor cells, and the like. However, the association between the LBX1 gene and scoliosis has not been known.

### SUMMARY OF THE INVENTION

An object of the present invention is to provide a method for accurately diagnosing the risk of onset of a bone and joint disease such as scoliosis and/or the presence or absence of onset of the same and a diagnosis reagent used for the method.

The present inventors conducted extensive and intensive investigations to solve the above-described problems and consequently identified that single nucleotide polymorphisms (SNPs) present in region 24 on the long arm of chromosome 10 (region 10q24) are associated with adolescent idiopathic scoliosis (AIS). Then, the present inventors found that the risk of onset of a bone and joint disease such as scoliosis and/or the onset of the same can be accurately estimated by analyzing these polymorphisms, thereby completing the present invention.

Specifically, the present invention includes the following aspects:

[1]

A method for diagnosing the risk of onset of a bone and joint disease and/or the presence or absence of onset of a bone and joint disease, said method comprising:

analyzing a single nucleotide polymorphism present in region 24 on the long arm of chromosome 10; and

diagnosing a bone and joint disease on the basis of a result of the analysis.

[2]

The method according to [1], wherein said bone and joint disease is scoliosis.

[3]

The method according to [1] or [2], wherein said single nucleotide polymorphism is a polymorphism of a nucleotide corresponding to the nucleotide at position 61 in a nucleotide sequence selected from SEQ ID NOS: 1 to 3 or a polymorphism of a nucleotide showing linkage disequilibrium with said nucleotide.

[4]

The method according to any one of [1] to [3], wherein said nucleotide showing linkage disequilibrium is a nucleotide corresponding to the nucleotide at position 61 in a nucleotide sequence selected from SEQ ID NOS: 4 to 11.

[5]

A probe for diagnosing a bone and joint disease, wherein said probe has a sequence of 10 or more nucleotides comprising the nucleotide at position 61 in a nucleotide sequence selected from SEQ ID NOS: 1 to 11 or has a complementary sequence thereof.

[6]

A primer for diagnosing a bone and joint disease, wherein said primer can amplify a region comprising the nucleotide at position 61 in a nucleotide sequence selected from SEQ ID NOS: 1 to 11.

By the present invention, the risk of onset (incidence risk) of a bone and joint disease such as scoliosis, which has conventionally been difficult to predict, can be accurately and simply predicted. In addition, onset of bone and joint disease such as scoliosis can be accurately and simply diagnosed. Therefore, the present invention contributes to the prevention and early treatment of bone and joint diseases such as scoliosis.

### BRIEF DESCRIPTION OF THE DRAWINGS

FIG. 1 is a view showing a linkage disequilibrium map of the region 10q24 (the upper panel) and results of association analysis with AIS (the lower panel). The linkage disequilibrium map was prepared on the basis of the Phase II HapMap (release 24) JPT data, in which SNPs genotyped in GWAS were boxed. The results of the association analysis are shown as −log₁₀ p values calculated by Cochran-Armitage trend test. In the results of the association analysis, the black diamonds represent the results of the GWAS and the white squares represent the results of imputation.

### MODES FOR CARRYING OUT THE INVENTION

### <1> Method of the Present Invention

The method of the present invention is a method for diagnosing the risk of onset of a bbne and joint disease and/or the presence or absence of onset of a bone and joint disease, comprising: analyzing a single nucleotide polymorphism present in region 24 on the long arm of chromosome 10 (region 10q24); and diagnosing a bone and joint disease on the basis of a result of the analysis. Specifically, in the present invention, the term “diagnosing” includes a diagnosis of the risk of onset of a bone and joint disease and a diagnosis of the presence or absence of onset of a bone and joint disease. In the method of the present invention, a result of analysis of an SNP is correlated with the risk of onset of a bone and joint disease and/or the presence or absence of onset of a bone and joint disease.

Examples of the bone and joint disease include, but not particularly limited to, a spinal disease, specifically scoliosis. The method of the present invention can be particularly used for the diagnosis of idiopathic scoliosis, whose etiology has not been conventionally specified. The scoliosis can be one developing in any stage of life, such as congenital, juvenile, adolescent, or adult scoliosis. For example, the scoliosis can be adolescent scoliosis, and specifically, can be adolescent idiopathic scoliosis (AIS).

The method of the present invention can be used for subjects of any race, and in particular, can be used for Asian subjects, such as Japanese and Chinese. In addition, the method of the present invention can be used for subjects of any sex.

Specific examples of the SNPS present in the region 10q24 include human rs11190870, rs625039, and rs11598564. Herein, the rs numbers represent registration numbers in the dbSNP database of the National Center for Biotechnology Information (http//www.ncbi.nlm.nih.gov/projects/SNP/). These three SNPs are located in a linkage disequilibrium block comprising LBX1 gene and a virtual gene FLJ41350 in region 10q24.31-q24.32. Accordingly, the bone and joint disease can be diagnosed, particularly, by analyzing an SNP present in this linkage disequilibrium block. Specific example of the LBX1 gene includes a sequence complementary to a region from 102986733 to 102988717 of GenBank Accession No. NC_{—}000010.10. Specific example of the virtual gene FLJ41350 includes a region from 102989351 to 102998616 of GenBank Accession No. NC_{—}000010.10.

rs11190870 refers to the polymorphism of thymine (T)/cytosine (C) of the nucleotide at position 21727733 in GenBank Accession No. NT_{—}030059.12. When the nucleotide is T, the possibility of the bone and joint disease or the risk of onset of the bone and joint disease is high. In addition, when analysis is made in consideration of genotype, the possibility of the bone and joint disease or the risk of onset of the bone and joint disease is high in the order of TT>TC>CC.

rs625039 refers to the polymorphism of guanine (G)/adenine (A) of the nucleotide at position 21742175 of GenBank Accession No. NT_{—}030059.12. When the nucleotide is G, the possibility of the bone and joint disease or the risk of onset of the bone and joint disease is high. In addition, when analysis is made in consideration of genotype, the possibility of the bone and joint disease or the risk of onset of the bone and joint disease is high in the order of GG>GA>AA.

rs11598564 refers to the polymorphism of adenine (A)/guanine (G) of the nucleotide at position 21713130 of GenBank Accession No. NT_{—}030059.12. When the nucleotide is G, the possibility of the bone and joint disease or the risk of onset of the bone and joint disease is high. In addition, when analysis is made in consideration of genotype, the possibility of the bone and joint disease or the risk of onset of the bone and joint disease is high in the order of GG>GA>AA.

Regarding the above-described three SNPs, the sequences of a total length of 121 bp, each of which comprises the SNP nucleotide and the 60 bp regions upstream and downstream thereof, were shown in SEQ ID NOS: 1 to 3. The nucleotide at position 61 in the sequence has the polymorphism.

In the present invention, a nucleotide corresponding to the above-described nucleotide is analyzed. The “nucleotide corresponding to the above-described nucleotide” refers to the corresponding nucleotide in the above-described region. That is, the expression “a nucleotide corresponding to the above-described nucleotide is analyzed” includes a case of analyzing the corresponding nucleotide in the above-described region even if the above-described sequence slightly changes at a position other than the SNP position due to a racial difference or the like.

Additionally, the nucleotide to be analyzed in the present invention is not limited to the above-described nucleotide, and a polymorphism of a nucleotide showing linkage disequilibrium with the above-described nucleotide can be analyzed. Herein, the “nucleotide showing linkage disequilibrium with the above-described nucleotide” refers to, for example, a nucleotide that satisfies a relationship of r²>0.5, r²>0.8, or r²>0.9 with the above-described nucleotide. The “r²” is a linkage disequilibrium coefficient. In addition, the nucleotide showing linkage disequilibrium with the above-described nucleotide can be identified, for example, by using the HapMap database (http://www.hapmap.org/index.html.ja) or the like. Alternatively, the nucleotide showing linkage disequilibrium with the above-described nucleotide can be identified by analyzing the sequences of DNAs extracted from a plurality of persons (usually, about 20 to 40 persons) and then screening a SNP showing linkage disequilibrium. When the analysis of the nucleotide showing linkage disequilibrium with the above-described nucleotide is made in consideration of genotype, the possibility of the bone and joint disease or the risk of onset of the bone and joint disease is high in the order of the homozygote of risk allele>the heterozygote of risk allele and non-risk allele>the homozygote of non-risk allele.

Examples of nucleotides showing linkage disequilibrium with rs11190870 at r²>0.5, include rs12771674, rs11598177, rs1407409, rs1322331, rs594791, rs679206, rs678741, and rs10883597, in addition to rs625039 and rs11598564 mentioned above. Regarding each of these nucleotides, the linkage disequilibrium coefficient (r²) to rs11190870, the combination of alleles, the risk allele, and the like are shown in Table 1. In the Table, the representations of alleles and risk alleles correspond to the forward strand of the NCBI reference sequence build 36. In addition, regarding rs12771674, rs11598177, rs1407409, rs1322331, rs594791, rs679206, rs678741, and rs10883597, the sequences of a total length of 121 bp, each of which comprises the SNP nucleotide and the 60 bp regions upstream and downstream thereof, were shown in SEQ ID NOS: 4 to 11, respectively. The nucleotide at position 61 in the sequence has the polymorphism.

TABLE 1
rs11190870 and SNPs showing linkage disequilibrium with
rs11190870 at r² > 0.5

| | rs11190870 | | Trend P | | | Risk |
|---|---|---|---|---|---|---|
| SNP | D′ | r² | GWAS | Imputation | Allele 1/2 | Allele |
| rs11598564 | 0.82 | 0.54 | 9.40E−08 | — | A/G | G |
| rs12771674 | 0.8 | 0.53 | — | — | A/G | A |
| rs11190870 | — | — | 1.27E−10 | — | T/C | T |
| rs11598177 | 1 | 0.76 | — | — | T/C | T |
| rs1407409 | 1 | 0.72 | — | 5.17E−10 | T/C | T |
| rs1322331 | 1 | 0.76 | — | 3.36E−08 | A/C | A |
| rs625039 | 1 | 0.69 | 4.75E−09 | — | G/A | G |
| rs594791 | 1 | 0.8 | — | — | C/T | C |
| rs679206 | 0.94 | 0.71 | — | 3.44E−07 | T/C | T |
| rs678741 | 0.94 | 0.7 | — | 4.34E−07 | A/G | A |
| rs10883597 | 0.93 | 0.6 | — | 2.01E−06 | C/T | C |

Allele 1 represents major allele.
Allele 1/2 is shown according to (+) strand of human reference sequence.

The bone and joint disease can be diagnosed by analyzing the type of the nucleotide of the above-described SNP and relating the obtained result to the bone and joint disease on the basis of the criteria as described above. One of the above-described SNPs can be analyzed solely or a plurality of SNPs including at least one of the above-described SNPs can be collectively analyzed (Haplotype Analysis). For example, a plurality of the above-described SNPs can be collectively analyzed, or at least one of the above-described SNPs can be analyzed in combination with known SNPs associated with the bone and joint disease (for example, Non-Patent Literature 12) or SNPs showing linkage disequilibrium with the known SNPs. By collectively analyzing a plurality of SNPs associated with the bone and joint disease, the accuracy of diagnosis of the bone and joint disease can be improved. For any SNP, either strand of double-stranded DNA can be analyzed. For example, regarding the sequence of the LBX1 gene, either the sense strand or antisense strand of the gene can be analyzed.

The sample used for the SNP analysis is not particularly limited as long as it is a sample containing the chromosomal DNA, and examples of such a sample include body fluids such as blood and urine, cells such as oral mucous membrane, and body hair such as hair on the head. These samples can be directly used for the SNP analysis, or the chromosomal DNA can be isolated from the samples by a conventional method and the isolated chromosomal DNA can be used for the analysis.

The SNP analysis can be carried out by a usual method for analyzing gene polymorphism. Examples of such a method include, but not limited to, sequencing analysis, PCR, hybridization, and invader assay.

Sequencing analysis can be carried out by a usual method. Specifically, sequencing reaction is performed using primers to be located at a position of several ten nucleotides on the 5′ side from a polymorphic nucleotide, and the type of the nucleotide at the corresponding position can be determined on the basis of the result of the analysis. In addition, before sequencing reaction, a fragment containing the SNP site can be preliminarily amplified by PCR or the like.

Also, the SNP analysis can be carried out by investigating the presence or absence of amplification by PCR. For example, primers which have sequences corresponding to a region containing a polymorphic nucleotide and whose 3′ ends correspond to the respective polymorphisms are prepared. PCR is performed using each primer, and the type of polymorphism can be determined on the basis of the presence of absence of an amplification product. Furthermore, the presence or absence of amplification can be detected by the LAMP method (Japanese Patent No. 3313358), the NASBA method (Nucleic Acid Sequence-Based Amplification; Japanese Patent No. 2843586), the ICAN method (Japanese Patent Application Laid-Open Publication No. 2002-233379), or the like. Other than these, a single-strand amplification method can also be used.

In addition, a DNA fragment containing a SNP site is amplified, and the type of polymorphism can be determined on the basis of a difference in electrophoretic mobility of amplification product. An example of such a method includes a PCR-SSCP (single-strand conformation polymorphism) method (Genomics. 1992 Jan. 1; 12(1): 139-146.). Specifically, at first, DNA containing a target SNP is amplified and the amplified DNA is dissociated into single-stranded DNAs. Next, the dissociated single-stranded DNAs are separated on a non-denaturing gel, and the type of polymorphism can be determined on the basis of the mobility difference between the separated single-stranded DNAs on the gel.

Furthermore, when a polymorphic nucleotide is contained in a restriction enzyme recognition sequence, analysis can be can be carried out on the basis of the presence or absence of cleavage by the restriction enzyme (RFLP method). In this case, at first, a DNA sample is cleaved by a restriction enzyme. Next, DNA fragment(s) are separated and the type of polymorphism can be determined on the basis of the size of the detected DNA fragment(s).

It is also possible to analyze the type of polymorphism by detecting the presence or absence of hybridization. Specifically, by preparing probes corresponding to the respective nucleotides and investigating which probe hybridizes to the DNA, the type of nucleotide of the SNP can be determined.

By determining the type of the nucleotide of an SNP, data for diagnosing the bone and joint disease can be obtained.

### <2> Diagnosis Reagent of Present Invention

The present invention also provides a diagnosis reagent, such as a primer or a probe, for diagnosing a bone and joint disease such as scoliosis. An example of such a probe includes a probe that contains the above-described SNP site and allows for the determination of the type of the nucleotide at the SNP site on the basis of the presence or absence of hybridization. Specific examples of the probe include a probe with a length of 10 or more nucleotides that has a sequence comprising the 61st nucleotide in a nucleotide sequence selected from SEQ ID NOS: 1 to 3 or has a complementary sequence thereof and a probe with the length of 10 or more nucleotides that has a sequence comprising a nucleotide showing linkage disequilibrium with the 61st nucleotide or has a complementary sequence thereof. The nucleotide sequence comprising “a nucleotide showing linkage disequilibrium with the nucleotide” and the regions upstream and downstream thereof is available, for example, from the dbSNP database of National Center for Biotechnology Information (http//www.ncbi.nlm.nih.gov/projects/SNP/). The length of the probe can be, for example, 15 to 35 nucleotides, or 20 to 35 nucleotides.

As well, examples of the primer include a primer usable in PCR for amplifying the above-described SNP site and a primer usable for sequencing analysis (sequencing) of the above-described SNP site. Specific examples of the primer include a primer capable of amplifying or sequencing a region comprising the 61st nucleotide in a nucleotide sequence selected from SEQ ID NOS: 1 to 3 and a primer capable of amplifying or sequencing a region comprising a nucleotide showing linkage disequilibrium with the 61st nucleotide. The length of primer can be, for example, 10 to 50 nucleotides, 15 to 35 nucleotides, or 20 to 35 nucleotides.

Examples of the primer for sequencing the above-described SNP site include a primer having a sequence of the 5′ side region of the above-described nucleotide, for example, a sequence of 30 to 100 nucleotides upstream of the above-described nucleotide, and a primer having a complementary sequence of the 3′ side region of the above-described nucleotide, for example, a complementary sequence of a region of 30 to 100 nucleotides downstream of the above-described nucleotide. Examples of the primer used to determine a polymorphism on the basis of the presence or absence of amplification by PCR include a primer that has a sequence comprising the above-described nucleotide and comprises the above-described nucleotide on the 3′ side of the primer, and a primer that has a sequence complementary to the sequence comprising the above-described nucleotide and comprises the nucleotide complementary to the above-described nucleotide on the 3′ side of the primer.

The diagnosis reagent of the present invention can include, in addition to the primer(s) and probe(s), polymerase and buffer for PCR, reagents for hybridization, and/or the like.

### EXAMPLES

Hereinafter, the present invention will be described in more detail with reference to Examples. However, the present invention is not limited thereto.

### Example 1

### Identification of SNPs Associated with Adolescent Idiopathic Scoliosis (AIS)

To identify genetic polymorphisms that determine AIS susceptibility, a genome-wide association study (GWAS) was carried out using Japanese subjects. GWAS is a genetic statistical method for screening genetic polymorphisms associated with phenotypes such as diseases. For example, genetic polymorphisms associated with a certain disease can be found by using SNPs at several hundred thousand to one million sites covering the whole human genome and statistically testing whether there are any difference in polymorphism frequencies between patients with the disease (cases) and subjects without the disease (controls).

### <Subjects>

Table 2 shows characteristics of the subjects used in the present Example.

TABLE 2
Characteristics of Japanese female subjects

| Cohort | Source | Platform | No. of subjects | Age | BMI |
|---|---|---|---|---|---|
| GWAS | | | | | |
| Case | 8 hospitals | Illumina | 1,033 | 17.5 (4.59) | 19.2 (2.24) |
| | | HumanHap610 | | | |
| Control | ORC ^{a} + BioBank | Illumina | 1,473 | 48.5 (15.2) | 22.1 (3.89) |
| | Japan ^{b} | HumanHap550 | | | |
| Replication | | | | | |
| Case | 8 hospitals | Invader assay | 326 | 18.4 (7.62) | 19.2 (2.33) |
| Control | BioBank Japan ^{c} | Illumina | 9,823 | 59.5 (13.2) | 22.8 (3.81) |
| | | Humanilap610 | | | |

ORC: Osaka-Midousuji Rotary Club.
^{a} The control subjects from ORC consisted of individuals with healthy volunteer.
^{b} The GWAS control subjects from BioBank Japan consisted of individuals with 13 diseases.
^{c} The replication control subjects from Biol3ank Japan consisted of individuals with 42 diseases.

Since AIS often occurs in females, the subjects recruited were all Japanese females. In the GWAS, 1,050 AIS subjects (cases) used were recruited from 8 hospitals, and in replication study, 326 AIS subjects (cases) used were recruited from the 8 hospitals. All the AIS subjects underwent clinical and radiographic examinations and were diagnosed as AIS by expert scoliosis surgeons. In all the cases, the Cobb angle, which is an indicator of scoliosis, was more than 20°. In the GWAS, 1,474 control subjects (controls) used consist of patients with diseases other than AIS registered in the BBJ and healthy volunteers recruited from the Osaka-Midousuji Rotary Club. In the replication study, 9,823 control subjects used were recruited from patients with diseases other than AIS registered in the BBJ.

The present study was approved by the Ethical Committee of the Institute of Medical Science, the University of Tokyo, and the Ethical Committee of the RIKEN Yokohama Institute, and informed consents were obtained from all the subjects and parents of some of the subjects.

### <GWAS>

Genotypes of the 1,050 AIS subjects were analyzed using Illumina Human 610 Genotyping BeadChip (Illumina, Inc., (USA)). Genotypes of the 326 control subjects were analyzed using Illumina Human 550 v3 Genotyping BeadChip (Illumina, Inc., (USA)). For quality control, the followings were excluded: SNPs with a call rate of below 0.99; SNPs whose p values for the Hardy-Weinberg equilibrium test are equal to or less than a cut-off value (P≦10⁻⁶); non-autosomal SNPs; monomorphic SNPs; SNPs not shared among the AIS subjects and the control subjects, SNPs with ambiguous calls, subjects with lower call rate in close relative pairs of identify-by-state (IBS)≧1.7 (16 AIS subjects; 1 control subject), and a subject determined as an outlier by principal component analysis (PCA) (1 AIS subject). Regarding 455,121 SNPs of 1,033 AIS subjects and 1,473 control subjects having passed through the quality control, the associations of the SNPs with AIS were evaluated by Cochran-Armitage trend test. The genomic inflation factor was 1.09, suggesting that the possibility of false positive associations due to population bias is low.

As a result of the GWAS, three SNPs present in the region 10q24 (i.e. rs11190870, rs625039, and rs11598564) satisfied P<1.10×10⁻⁷ (=0.05/455,121) set as a threshold of genome-wide significance and showed significant associations with AIS (the column of “GWAS” in Table 3). This threshold corresponds to P<0.05 after Bonferroni corrections based on 455,121 times of tests.

TABLE 3
SNPs associated with adolescent idiopathic scoliosis

| | | | Case | | | | Control | | | | | | |
|---|---|---|---|---|---|---|---|---|---|---|---|---|---|
| | Allele1/2 | | Genotype count | | | | Genotype count | | | | | Odds ratio^{b} | |
| dbSNP ID | Risk | Study | 11 | 12 | 22 | RAF | 11 | 12 | 22 | RAF | P value^{a} | (95% CI) | Pₕₑᵢ^{c} |
| rs11190870 | T/C | GWAS | 449 | 470 | 114 | 0.662 | 479 | 728 | 266 | 0.572 | 1.27.E−10 | 1.46 (1.30-1.65) | |
| | T | Replication | 152 | 148 | 26 | 0.693 | 3129 | 4809 | 1883 | 0.563 | 5.13.E−11 | 1.75 (1.48-2.07) | |
| | | Combined^{d} | 601 | 618 | 140 | 0.670 | 3608 | 5537 | 2149 | 0.565 | 1.24.E−19 | 1.56 (1.41-1.71) | 0.0881 |
| rs625039 | G/A | GWAS | 533 | 424 | 76 | 0.721 | 600 | 695 | 178 | 0.643 | 4.75.E−09 | 1.43 (1.27-1.62) | |
| | G | Replication | 172 | 135 | 19 | 0.735 | 1297 | 4579 | 3947 | 0.635 | 1.69.E−07 | 1.59 (1.34-1.90) | |
| | | Combined^{d} | 705 | 559 | 95 | 0.724 | 1475 | 5274 | 4547 | 0.636 | 4.30.E−15 | 1.49 (1.35-1.65) | 0.341 |
| rs11598564 | A/G | GWAS | 297 | 508 | 228 | 0.533 | 310 | 724 | 439 | 0.456 | 9.40.E−08 | 1.36 (1.22-1.53) | |
| | G | Replication | 107 | 156 | 63 | 0.567 | 2107 | 4837 | 2879 | 0.461 | 8.82.E−08 | 1.54 (1.31-1.80) | |
| | | Combined^{d} | 404 | 664 | 291 | 0.542 | 2417 | 5531 | 3318 | 0.46 | 5.98.E−14 | 1.42 (1.30-1.56) | 0.226 |

Allele 1 represents major allele.
Allele 1/2 is shown according to (+) strand of human reference sequence.
Risk: risk allele.
RAF: risk allele frequency.
CI: confidence interval.
^{a}Chromosomal location based on assembly GRCh37, build 37.1.
^{b}P value of the Cochran-Armitage trend test.
^{c}Allelic odds ratio with 95% CI.
^{d}P value of the Breslow-Day test.
^{e}calculated by the Mantel-Haenzel method.

### <Replication Study>

To confirm the associations of these three SNPs with AIS, replication study was carried out using subjects independent of those analyzed in the GWAS. Genotypes of the 326 AIS subjects were analyzed by multiplex-PCR invader assay (Third Wave Technologies). Genotypes of the 9,823 control subjects were analyzed using the Illumina Human 550 v3 Genotyping BeadChip (Illumina, Inc., (USA)).

As a result of the replication study, all the three SNPs satisfied P<1.67×10⁻² (=0.05/3), which was the threshold of significance after Bonferroni corrections, and showed significant associations with AIS (the column of “Replication” in Table 3). In addition, the p values for the Breslow-Day test were all 0.05 or less (the column of “Pₕₑₜ” in Table 3), and hence, no significant heterogeneity was observed between the GWAS subjects and the replication study subjects.

In both of the GWAS and the replication study, the SNP that showed the strongest association with AIS was rs11190870. As a result of combined analysis of the GWAS and the replication study using Mantel-Haenszel method, rs11190870 showed association with AIS at P=1.24×10⁻¹⁹ (odds ratio=1.56) (the column of “Combined” in Table 3).

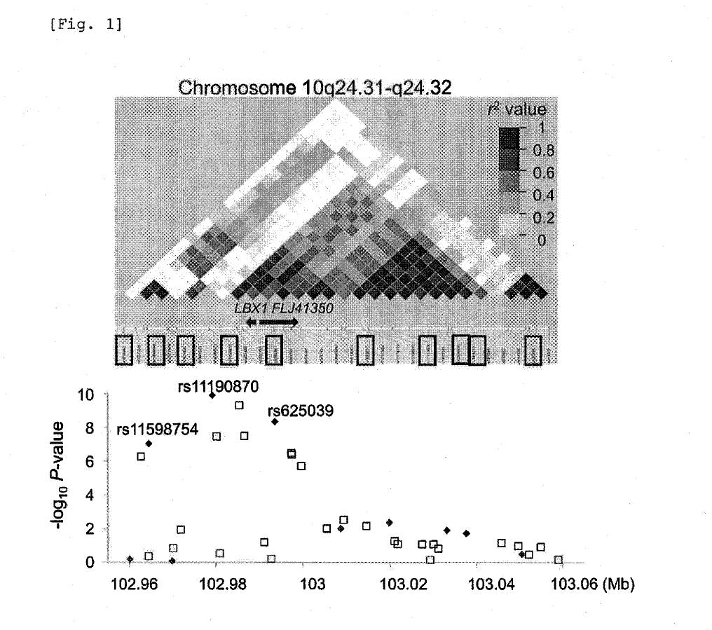

A linkage disequilibrium map of the region 10q24 and results of association analysis are shown in Table 1. All of rs11190870, rs625039, and rs11598564 were located in a linkage disequilibrium block of approximately 80 kb comprising the LBX1 gene and the virtual gene FLJ41350 in the region 10q24.31-q24.32, and more specifically, rs11190870 was located in the 3′ flanking region of the LBX1 gene (FIG. 1). In addition, as a result of haplotype analysis using these three SNPs, there was found no haplotype showing stronger association with AIS than rs11190870.

Although genome-wide linkage analyses have identified predisposing loci of AIS, there has been no report on region 10q24 (Non-patent Literature 1 and 2). In addition, in a TDT-based GWAS, among the above three SNPs (rs11190870, rs625039, and rs11598564), rs11598564 was included in the top 100 SNPs associated with AIS, but the p value for the TDT analysis resulted in P=8.19×10⁻⁵ (all races) or P=1.03×10⁻³ (non-Hispanic), and hence, there was observed no significant association (Non-patent Literature 12). There have been no findings reporting on genes or single nucleotide polymorphisms (SNPs) whose associations with AIS have been confirmed at a genome-wide level. Accordingly, the present invention has discovered SNPs significantly associated with AIS at the genome wide level for the first time.

The LBX1 gene has been cloned as a homeobox gene, and there have been reports on the expression of the gene in dorsal spinal cord, hindbrain, a part of cardiac neural crest, muscle precursor cells, and so forth, in vertebrates. The present inventor also confirmed the expression of the LBX1 gene in spinal cord and the skeletal muscle. Regarding mice, it is known that Lbx1 protein acts as an identity determinant of dorsal spinal neurons and somatosensory neurons in the hindbrain. The dorsal spinal cord is involved in the transmission of somatosensory information, and patients with scoliosis such as AIS often show abnormal somatosensory function (Guo, X. et al. Spine 31, E437-E440 (2006)). Also, in patients with spastic diplegea, the incidence rate of scoliosis is known to increase after dorsal rhizotomy (Steinbok, P. et al. J. Neurosurg. 102, 363-373. (2005)). Accordingly, the LBX1 gene may be implicated in the etiology of scoliosis through abnormalities in somatosensory function.

### <Imputation>

In addition, for further analysis of the association of the region 10q24 with AIS, the genotypes of other SNPs (those that had not been analyzed in the GWAS) present in the above-mentioned linkage disequilibrium block comprising rs11190870 were analyzed by MACH (http://www.sph.umich.edu/csg/yli/mach/index.html) thereby to evaluate the associations with AIS. As a result of the imputation, three more SNPs were newly found to satisfy P<1.10×10⁻⁷ set as the threshold of genome-wide significance and show significant associations with AIS (white squares in the lower panel of FIG. 1). These three SNPs were located in the 3′ flanking region of the LBX1 gene and all of them showed linkage disequilibrium with rs11190870 at r²>0.7. The associations of the three SNPs with AIS were weaker than the association of rs11190870 with AIS.

### <Association Analysis Using Male Subjects>

Regarding the three SNPs (rs11190870, rs625039, and rs11598564) showing strong associations with AIS in the female subjects, the associations with AIS in male subjects were verified. Based on the same criteria as those for the female subjects, 94 male AIS subjects and 1,849 male control subjects were recruited. Genotypes of the AIS subjects were analyzed by multiplex-PCR invader assay (Third Wave Technologies). Genotypes of the control subjects were analyzed using Illumina HumanHap 550 v3 Genotyping BeadChip (Illumina, Inc., (USA)). The associations of the above three SNPs with AIS were evaluated by Cochran-Armitage trend test. As a result, in all the three SNPs, the associations with AIS were replicated (Table 4). According to the odds ratios, the association of rs11190870 with AIS was stronger than that in the female population, and the associations of rs625039 and rs11598564 with AIS were comparative to those in the female population. Accordingly, it was revealed that rs11190870, rs625039, and rs11598564 are AIS-susceptible polymorphisms also in males and hence can be used also for the diagnosis of AIS in males.

TABLE 4
Associations of SNPs with adolescent idiopathic scoliosis in male Japanese

| | Case | | | | Control | | | | | |
|---|---|---|---|---|---|---|---|---|---|---|
| | Genotype count | | | | Genotype count | | | | | Odds ratio^{b} |
| dbSNP ID | 11 | 12 | 22 | RAF | 11 | 12 | 22 | RAF | P value^{a} | (95% CI) |
| rs11190870 | 49 | 38 | 7 | 0.723 | 574 | 882 | 393 | 0.54895 | 4.11.E−06 | 2.15 (1.55-2.98) |
| rs625039 | 56 | 31 | 7 | 0.761 | 726 | 848 | 275 | 0.62196 | 1.56.E−04 | 1.93 (1.37-2.72) |
| rs11598564 | 27 | 48 | 19 | 0.543 | 391 | 866 | 592 | 0.44565 | 1.10.E−02 | 1.48 (1.10-1.98) |

RAF: risk allele frequency.
CI: confidence interval.
^{a}P value of the Cochran-Armitage trend test.
^{b}Allelic odds ratio with 95% CI.

### Example 2

### Verification of SNPs Associated with AIS

In the present Example, regarding rs11190870 having shown the strongest association with AIS in Example 1, verification was carried out using Chinese subjects.

### <Subjects>

From the Duchess of Kent Children's Hospital in Hong Kong, 300 AIS subjects (cases) were recruited. Recruitment criteria were as follows: (1) Subjects with idiopathic scoliosis with Cobb angle of greater than 35° and in need of surgery; (2) The onset of scoliosis was after 10 years and under 20 years of age; and (3) Patients with neuromuscular scoliosis, patients with congenital scoliosis, and patients with syndromal scoliosis were excluded.

From a data set of a southern Chinese population of 3,500 subjects recruited in a genetic study of degenerative disc disease, 788 control subjects (controls) were randomly selected and used in the verification. MRI assured that none of the subjects of the data set had AIS.

Informed consents were obtained from all the subjects.

### <Genotyping>

Genotyping of rs11190870 was carried out using PCR-based invader assay (Ohnishi, Y. et al. J. Hum. Genet. 46, 471-477 (2001)). Probes were designed and synthesized using Third Wave. The invader assay plates were read using the ABI PRISM 7900HT sequence detection system (Applied Biosystems).

### <Statistical Analysis>

The association of rs11190870 with AIS was analyzed by Cochran-Armitage trend test. Hardy-Weinberg equilibrium of genotype frequency was analyzed by Chi-square test.

### <Results>

The analysis results of the total cases and controls (300 cases and 788 controls) and the analysis results of female only cases and controls (248 cases and 489 controls) are shown in Table 5. The genotype frequency of rs11190870 in the control group followed Hardy-Weinberg equilibrium (total controls: P=0.993; female only controls: P=0.756). The frequency of allele T as risk allele in the total cases was significantly higher than that in the total controls (P=9.1×10⁻¹⁰; OR=1.85; 95% CI: 1.52-2.25). Additionally, the frequency of allele T as risk allele in the female only cases was significantly higher than that in the female only controls (P=5.1×10⁻⁸; OR=1.87; 95% CI: 1.49-2.35). These ORs were close to the OR in the Japanese population (OR=1.56), thus suggesting that the effect size of the risk allele was similar between the Japanese population and the southern Chinese population. Additionally, risk allele frequencies in the southern Chinese population (cases: 0.67; controls: 0.52) were extremely close to those in the Japanese population (cases: 0.67; controls: 0.52). Furthermore, the statistical power was greater than 99% in both of the total group and the female only group. Accordingly, the association of rs11190870 with AIS was replicated and confirmed also in the southern Chinese population.

TABLE 5
Association of rs11190870 with adolescent idiopathic scoliosis in southern Chinese

| | | Genotype count^{a} | | RAF^{b} | | | |
|---|---|---|---|---|---|---|---|
| dbSNP ID | Sex | Case | Control | Case | Control | P^{c} | OR^{d} |
| rs11190870 | Total | 133/136/31 | 216/393/179 | 0.67 | 0.52 | 9.1 × 10⁻¹⁰ | 1.85 (1.52-2.25) |
| | Female | 114/109/25 | 136/247/106 | 0.68 | 0.53 | 5.1 × 10⁻⁸ | 1.87 (1.49-2.35) |

^{a}Number of [homozygotes of risk allele, TT]/[heterozygotes, TC]/[homozygotes, of the other allele, CC].
^{b}Risk allele (T-allele) frequency.
^{c}The Cochran-Armitage trend test.
^{d}Allelic odds ratios with 95% confidence interval.

### Example 3

### Meta-Analysis

Meta-analysis was carried out by combining the results of Examples 1 and 2. As a result, the combined total southern Chinese and Japanese samples showed P=4.8×10⁻²⁴ and OR=1.58, and the combined female only samples showed P=1.4×10⁻²² and OR=1.58, suggesting that rs11190870 is extremely strongly associated with AIS.

All the documents cited herein is incorporated by reference as a part of this application.

### INDUSTRIAL APPLICABILITY

Accordingly, the case-control association analysis at the whole genome level identified the AIS-associated region at the genome-wide level. The SNPs present in the region are useful in the diagnosis of a bone and joint disease such as scoliosis and contribute to the prevention and/or treatment thereof.

1. A method for diagnosing the risk of onset of a bone and joint disease and/or the presence or absence of onset of a bone and joint disease, said method comprising:
analyzing a single nucleotide polymorphism present in region 24 on the long arm of chromosome 10; and
diagnosing a bone and joint disease on the basis of a result of the analysis.

2. The method according to claim 1, wherein said bone and joint disease is scoliosis.

3. The method according to claim 1, wherein said single nucleotide polymorphism is a polymorphism of a nucleotide corresponding to the nucleotide at position 61 in a nucleotide sequence selected from SEQ ID NOS: 1 to 3 or a polymorphism of a nucleotide showing linkage disequilibrium with said nucleotide.

4. The method according to claim 1, wherein said nucleotide showing linkage disequilibrium is a nucleotide corresponding to the nucleotide at position 61 in a nucleotide sequence selected from SEQ ID NOS: 4 to 11.

5. A probe for diagnosing a bone and joint disease, wherein said probe has a sequence of 10 or more nucleotides comprising the nucleotide at position 61 in a nucleotide sequence selected from SEQ ID NOS: 1 to 11 or has a complementary sequence thereof.

6. A primer for diagnosing a bone and joint disease, wherein said primer can amplify a region comprising the nucleotide at position 61 in a nucleotide sequence selected from SEQ ID NOS: 1 to 11.


SEQUENCE LISTING (11 sequences)

SEQ ID NO: 1 - DNA, 121 bp, Homo sapiens
cccacaccccgccccggccctccaacaccagaggaattatcaactagaatttgattaataygcaaatcgcaggcaaacagctccatataattctttcagtggagagtaattaatcaccagt

SEQ ID NO: 2 - DNA, 121 bp, Homo sapiens
aggtgggcacatttttctgggagcaatattatataacacccaggattctcaaagcgggtgrggatccagaaaaggtaaaggctggaatcaggccactgtagggaagggaggcaagccactg

SEQ ID NO: 3 - DNA, 121 bp, Homo sapiens
agactcatacctgtggatagaggagaggtctagcgccgaacttctccaacttcaatgcagrcaagaagctctcaggaaaatctcattaacatgtggatactgactcaacaggcctaggtgg

SEQ ID NO: 4 - DNA, 121 bp, Homo sapiens
ctgtcatctgcgatcagcctcaggtaaacctaggtccacgccgagccttggtctgctctcrccacctgggactccctgagggccctacagtgttactctccatcttcatcctcttgggctc

SEQ ID NO: 5 - DNA, 121 bp, Homo sapiens
gttgggtgtgggcctgtgggcctgtctgggcttacccttcatttccatgtggacatgcagycccagggagacctcaggcagatggatcaataggccccattatagacatcaggagagagga

SEQ ID NO: 6 - DNA, 121 bp, Homo sapiens
cctcatccttggggagtcgggggccaaaggctaaaggcatcaaggccaggctgaatttcartgtgggcacctggaactgtaagcccaagcgtctgggagctggtgtgaatttctggcaaca

SEQ ID NO: 7 - DNA, 121 bp, Homo sapiens
gtttgctgagggcgagacagaaaccagggcggggaacactaaccctgcggtctaggggagkggcagctccggctgttttgatttttctctgcatgtggcgaatgcaccggcccgctccttc

SEQ ID NO: 8 - DNA, 121 bp, Homo sapiens
agggtgaaacccttagtctcttgtttatcggcagcaggtctgcagcctagagccaggggcygctggactcctgggatggctatttacttatttatggggtggggtgaggggtggggatggg

SEQ ID NO: 9 - DNA, 121 bp, Homo sapiens
ttcgcagagacagatgaggtgggctggcgattccctagcctcagatggctctgccaccagycagctctgtgatcctgggtaagtctttcaacctctctgagtcttgacgttggtaaaaggc

SEQ ID NO: 10 - DNA, 121 bp, Homo sapiens
gagaaggtgttgggagactaaatcttttcattcgtttgagaaagggagtcgcctgtgttgrgacctaaggaggaagtgaggttcttgagaattctagatctgaaatggccgtttgttttaa

SEQ ID NO: 11 - DNA, 121 bp, Homo sapiens
agtcatgcaggacaggcttgaagtgggcaggtgagtctccctggctagaatccatactctyctaggaatctcagtcttcctctgcccccatcaccccatccctgggagtgggggctgcaca